(19) United States
(12) Patent Application Publication
(10) Pub. No.: US 2009/0035231 A1
(43) Pub. Date: Feb. 5, 2009

(54) TOPICAL ORAL COMPOSITION

(76) Inventor: Irwin E. Smigel, New York, NY (US)

Correspondence Address: MEREDITH & KEYHANI, PLLC, 330 MADISON AVE., 6TH FLOOR, NEW YORK, NY, 10017, US

(21) Appl. No.: 11/831,080

(22) Filed: Jul. 31, 2007

(51) Int. Cl. A61K 8/22; A61Q 11/00

(52) U.S. Cl. 424/53

(57) ABSTRACT

A topical oral composition in an orally acceptable carrier, the composition comprising: dehydroacetic acid, a peroxide source and optionally sodium lauryl sulfoacetate, gelling agents, flavors, and abrasives.

The present invention relates generally to topical oral compositions, and in particular to germicidal oral compositions.

The number of dental plaque micro-organisms use to colonize the enamel tooth surface have been well characterized. The normal oral cavity contains at least 300 different bacterial species. The great majority of these are found in dental plaque. Organisms such as oral streptococci sanguis and streptococcus parasanguis are among the earliest plaque colonizing organisms. Also streptococcus mutans is the major cause of dental caries due to both sucrose-independent and sucrose-dependent mechanisms of colonization and the ability to produce large amounts of lactic acid responsible for demineralizing the enamel surface creating a carious lesion. Lesions in turn will ease formation of stains on the enamel of the teeth. Therefore, the inhibition of these three species is critical in reducing dental plaque and caries and in turn easing the removal of stains of variety in nature.

Accordingly, what is needed is a topical oral composition that inhibits the colonization of bacterial species.

### SUMMARY OF THE INVENTION

The present invention relates generally germicidal topical oral compositions.

According to one embodiment of the present invention, a topical oral composition in an orally acceptable carrier is provided, the composition comprising: sodium lauryl sulfoacetate, dehydroacetic, and a peroxide source

According to another embodiment of the present invention, a topical oral composition in an orally acceptable carrier is provided, the composition comprising: sodium lauryl sulfoacetate, between 0.1 and 1.0 dehydroacetic acid, and a peroxide source selected from the group consisting of hydrogen peroxide, urea hydrogen peroxide and calcium peroxide.

These and other features, aspects and advantages of the present invention will become better understood with reference to the following description and claims.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The term topical oral composition refers to a product which in the ordinary course of usage is not intentionally swallowed, but rather is retained in the oral cavity for a time sufficient to contact dental surfaces and oral tissue. The term topical oral composition is intended to include, by way of example, toothpaste (gel or paste), tooth powder, denture gel, denture product, mouthrinse, mouthspray, foam and dentrifice. According to the present invention, a topical oral composition in an orally acceptable carrier is provided, the composition comprising: sodium lauryl sulfoacetate, dehydroacetic acid, and a peroxide source. There may also be gelling agents, flavors and abrasives. By way of example, gelling agents may be The peroxide source may be hydrogen peroxide, urea hydrogen peroxide, calcium peroxide and any combination thereof. It is desired that the dehydroacetic acid ranges from 0.05 to 30.0 percent by weight and is preferably between 0.1 and 1.0 percent by weight. The peroxide source may be between 0.005 and 30 percent by weight. The Ratio of dehydroacetic acid to the peroxide source may be 0.05% to 1.00% of dehydroacetic acid to 0.01% to 1.00% of the peroxide source. The sodium lauryl sulfoacetate may be between 0.1 and 10.0 percent by weight. The ph of the topical oral composition may be between 3.0 and 9.0 and preferably between 6.5 and 8.5. The composition may be further comprised of malic acid.

TABLE 1

| | EXAMPLE | EXAMPLE | EXAMPLE |
|---|---|---|---|
| COMPONENT | 1 | II | III |
| Deionized water | 21.34% | 63.79% | 76.72% |
| Sorbitol (70%) | 20% | 8% | 5% |
| Glycerine (99.7%) | 20% | 8% | 5% |
| Urea Hydrogen Peroxide | 3% | 1.5% | 1% |
| Calcium Peroxide | 2.5% | 1.25% | 0.8% |
| Sodium Perborate | 1.3% | 1.15% | 0.4% |
| Sodium Bicarbonate | 10% | 5% | 3.33% |
| Tetra Sodium Pyrophosphate | 3% | 1.5% | 1% |
| Corn Starch | 1% | 0.5% | 0.3% |
| Sodium Saccharin | 0.5% | 0.25% | 0.2% |
| Methyl Paraben | 0.5% | 0.25% | 0.2% |
| Propyl Paraben | 0.1% | 0.05% | 0.03% |
| Sodium Mono | 0.76% | 0.76% | 0.76% |
| Fluorophosphate | | | |
| Dehydroacetic Acid | 1% | 0.5% | 0.33% |
| Malic Acid | 5% | 2.5% | 1.7% |
| Cellulose Gum | 2% | 1% | 0.6% |
| Titanium Dioxide | 4% | 2% | 1.3% |
| Sodium Lauryl Sulfoacetate | 3% | 1.5% | 1% |
| Flavor | 1% | 0.5% | 0.33% |

Table 1 provides examples according to the present invention. As shown, according to one example Example 1: deionized water is 21.34 percent by weight, sorbitol (70%) is 20% by weight, glycerine (99.7%) is 20 percent by weight, urea hydrogen peroxide is three (3) percent by weight, calcium peroxide is 2.5 percent by weight, sodium perborate is 1.3 percent by weight, sodium bicarbonate is 10 percent by weight, tetra sodium pyrophosphate is 3 percent by weight, corn starch is 1 percent by weight, sodium saccharin is 0.5 percent by weight, methyl paraben is 0.5 percent by weight, propyl paraben is 0.1 percent by weight, sodium mono fluorophosphate is 0.76 percent by weight, dehydroacetic acid is 1 percent by weight, malic acid is 5 percent by weight, cellulose gum is 2 percent by weight, titanium dioxide is 4 percent by weight, sodium lauryl sulfoacetate is three percent by weight, and flavor is one percent by weight.

According to the present invention and as shown in Table 1, is Example 2. Example 2: deionized water is 63.79 percent by weight, sorbitol (70%) is 8% by weight, glycerine (99.7%) is 8 percent by weight, urea hydrogen peroxide is 1.5 percent by weight, calcium peroxide is 1.25 percent by weight, sodium perborate is 1.15 percent by weight, sodium bicarbonate is 5 percent by weight, tetra sodium pyrophosphate is 1.5 percent by weight, corn starch is 0.5 percent by weight, sodium saccharin is 0.25 percent by weight, methyl paraben is 0.25 percent by weight, propyl paraben is 0.05 percent by weight, sodium mono fluorophosphate is 0.76 percent by weight, dehydroacetic acid is 0.5 percent by weight, malic acid is 2.5 percent by weight, cellulose gum is 1 percent by weight, titanium dioxide is 2 percent by weight, sodium lauryl sulfoacetate is 1.5 percent by weight, and flavor is 0.5 percent by weight.

Another example, according to the present invention and as shown in Table 1, is Example 3. Example 3: deionized water is 76.72 percent by weight, sorbitol (70%) is 5% by weight, glycerine (99.7%) is 5 percent by weight, urea hydrogen peroxide is 1 percent by weight, calcium peroxide is 0.8 percent by weight, sodium perborate is 0.4 percent by weight, sodium bicarbonate is 3.33 percent by weight, tetra sodium pyrophosphate is 1 percent by weight, corn starch is 0.3 percent by weight, sodium saccharin is 0.2 percent by weight, methyl paraben is 0.2 percent by weight, propyl paraben is 0.03 percent by weight, sodium mono fluorophosphate is 0.76 percent by weight, dehydroacetic acid is 0.33 percent by weight, malic acid is 1.7 percent by weight, cellulose gum is 0.6 percent by weight, titanium dioxide is 1.3 percent by weight, sodium lauryl sulfoacetate is 1 percent by weight, and flavor is 0.33 percent by weight.

It should be understood that the foregoing relates to preferred embodiments of the invention and that modifications may be made without departing from the spirit and scope of the invention as set forth in the following claims.

I claim:

1. A topical oral composition in an orally acceptable carrier, said composition comprising:
dehydroacetic acid, sodium lauryl sulfoacetate and a peroxide source.

2. A topical oral composition in an orally acceptable carrier as in claim 1, further comprising a gelling agent.

3. A topical oral composition in an orally acceptable carrier as in claim 1, further comprising flavors.

4. A topical oral composition in an orally acceptable carrier as in claim 1, further comprising abrasives.

5. A topical oral composition in an orally acceptable carrier as in claim 1, wherein said peroxide source is at least one peroxide source selected from the group consisting of hydrogen peroxide, urea hydrogen peroxide and calcium peroxide.

6. A topical oral composition in an orally acceptable carrier as in claim 1, wherein said dehydroacetic acid is between 0.05 to 30.0 percent by weight.

7. A topical oral composition in an orally acceptable carrier as in claim 6, wherein said dehydroacetic acid is between 0.1 and 1.0 percent by weight.

8. A topical oral composition in an orally acceptable carrier as in claim 1, wherein said peroxide source is between 0.005 and 30 percent by weight.

9. A topical oral composition in an orally acceptable carrier as in claim 1, wherein said sodium lauryl sulfoacetate is between 0.1 and 10.0 percent by weight.

10. A topical oral composition in an orally acceptable carrier as in claim 1, wherein said sodium lauryl sulfoacetate is preferably between 0.5 and 3.0 percent by weight.

11. A topical oral composition in an orally acceptable carrier as in claim 1, wherein said topical oral composition has a pH between 3.0 and 9.0.

12. A topical oral composition in an orally acceptable carrier as in claim 1, wherein said topical oral composition has a pH preferably between 6.5 and 8.5.

13. A topical oral composition in an orally acceptable carrier as in claim 1, further comprising malic acid.

14. A topical oral composition in an orally acceptable carrier as in claim 1, wherein the Ratio of dehydroacetic acid to said peroxide source is 0.05% to 1.00% of dehydroacetic acid to 0.01% to 1.00% of said peroxide source.

15. A topical oral composition in an orally acceptable carrier, said composition comprising:
sodium lauryl sulfoacetate, between 0.1 and 1.0 dehydroacetic acid, and at least two peroxide sources, each of said peroxide sources selected from the group consisting of hydrogen peroxide, urea hydrogen peroxide and calcium peroxide.

16. A topical oral composition in an orally acceptable carrier as in claim 15, further comprising a gelling agent.

17. A topical oral composition in an orally acceptable carrier as in claim 15, further comprising flavors.

18. A topical oral composition in an orally acceptable carrier as in claim 15, further comprising abrasives.

19. A topical oral composition in an orally acceptable carrier as in claim 15, wherein said peroxide source is between 0.005 and 30 percent by weight.

20. A topical oral composition in an orally acceptable carrier as in claim 15, wherein said sodium lauryl sulfoacetate is between 0.1 and 10.0 percent by weight.

21. A topical oral composition in an orally acceptable carrier as in claim 15, wherein said topical oral composition has a pH between 3.0 and 9.0.

22. A topical oral composition in an orally acceptable carrier as in claim 15, wherein said topical oral composition has a pH preferably between 6.5 and 8.5.

23. A topical oral composition in an orally acceptable carrier as in claim 15, further comprising malic acid.

24. A topical oral composition in an orally acceptable carrier as in claim 15, wherein the Ratio of dehydroacetic acid to said peroxide source is 0.05% to 1.00% of dehydroacetic acid to 0.01% to 1.00% of said peroxide source.
